(19) United States
(12) Patent Application Publication
(10) Pub. No.: US 2005/0107750 A1
(43) Pub. Date: May 19, 2005

(54) Arterio-seal sheath with ots(option-to-seal)

(76) Inventor: Mark Gelido Barongan, San Diego, CA (US)

Correspondence Address: Mr. Mark Gelido Barongan, 6161 El Cajon Blvd. #470, San Diego, CA, 92115, US

(21) Appl. No.: 10/962,280

(22) Filed: Oct. 9, 2004

Related U.S. Application Data
(60) Provisional application No. 60/510,500, filed on Oct. 14, 2003.

(51) Int. Cl. A61M 5/00

(52) U.S. Cl. 604/264

(57) ABSTRACT

A system to beginning an intravascular procedure (i.e., angioplasty, atherectomy, stenting, angiography, venous filtration, liquid infusion, dialysis, intravascular ultrasound process, and the like) and a sealing of a wound site with reduced number of exchanges in procedure related devices. Moreover, the sealing means assure complete sealing and reduction of chances of bacterial infection of wound site.

### CROSS-REFERENCE TO RELATED APPLICATIONS

This application claims priority to Provisional Application No.: 60/510,500 filed Oct. 14, 2003.

### FEDERALLY SPONSORED RESEARCH

Not Applicable

### BACKGROUND OF THE INVENTION

This invention is for sealing puncture wounds in blood vessels that are often a result of intervening medical procedures. Presently, there are numerous percutaneous puncture sealing devices available:
U.S. Pat. No. 5,383,896 to Gershony et al. (1995) is a device that includes a shaft section with an expandable balloon and atraumatic tip at its distal end. Once the balloon is placed within the blood vessel, a form of gas will be injected through the proximal end though the inflation/deflation port. A fixation collar is loaded onto the shaft of the device to secure the placement of the balloon. This device, however, is seldom used in the field.

U.S. Pat. No. 5,391,183 to Janzen et al. (1995) is a vascular hemostatic device, the Vasoseal, which inserts collagen through a tissue channel and against the outside wall of a blood vessel to seal the puncture. This device, however, often fails to completely seal the wound site, thus causes bleeding. Therefore, manual pressure must be applied to the site, which takes about twenty minutes for a patient that does not have blood thinning medication, and considerably more time for one that has. The other problem with having a considerable amount of collagen in the body is that if the collagen does not dissipate equally, a little ball or olive pit sized lump remains under the skin. The dissolving time for these lumps is between 4-6 weeks. However, if the body does not dissolve it, the body may surround it with calcium, an eggshell-like effect, around the lump in order to protect the body from “non-self” material.

U.S. Pat. No. 5,645,566 to Brenneman et al. (1997) is a device to facilitate the access of sealants to percutaneous puncture sites. Sealants may be in the form of an inflatable balloon, a collapsible prong assembly, or a resilient foam pad. The foam pad is biodegradable. This device, however, is seldom used in the field.

U.S. Pat. No. 5,690,674 to Diaz (1997) is an elastic, biodegradable plug used to seal punctures. The plug consists of two retainers attached together. Once the distal retainer is fitted inside the blood vessel, the other retainer is positioned on the outside of the vessel, creating a sandwich effect between the retainers and the vessel wall. This device, however, is seldom used in the field.

U.S. Pat. No. 5,728,134 to Barak (1998) is a method and apparatus for sealing artery punctures by a balloon after arterial catheterization. The balloon seals blood flow from the puncture after being inflated by a form of gas. This device, however, is seldom used in the field.

U.S. Pat. No. 6,090,130 to Nash et al. (2000) the Angio-Seal system, which comprises of a percutaneous puncture sealing device, a component of the system, that consists a radiopaque rigid anchor to use within the blood vessel and a compressed collagen plug to use within the puncture tract leading to the vessel. This device is considered to be one of the most effective sealing devices on the market today. The drawback of this system is that the sealing process is cumbersome, requiring the exchange of sheath and guide-wire to that of the Angio-Seal. This exchange process often leads to more bleeding. When excessive blood leaks into the surrounding tissue from the exchanging process, the gauging of depth for the device changes. Excess blood in the tissue track can give a false indication of being in the true artery or blood vessel. The blood coming from the tissue track would be the blood coming out from the port to indicate one is in the artery. This would be perceived to be a positive confirmation when in fact it would be a false confirmation. To compensate for this possible oversight, a user may advance the device in farther than normal to ensure the confirmation is true. A drawback to additional advancements of the Angio-Seal can cause the radiopaque rigid anchor not to expel from the carrier tube. In addition, there are still chances of bleeding of the wound site because the collagen mesh would not seal the wound site on top and the rod-like shape of the anchor is unable to completely cover the wound opening from the inside, thus the chance of bleeding is likely to occur. If this happens, manual pressure or compression to the site is needed.

U.S. Pat. No. 6,126,675 to Shchervinsky et al. (2002) is a sealing device consisting of an elongated tube with one closed and flat end to seal the puncture. Above the flat end of the tube is an expandable region. A bio-absorbable mesh surrounds the tube. The puncture is sealed by the sandwiching action between the expandable region, the vessel wall, and the flat end. This device, however, is seldom used in the field.

U.S. Pat. No. 6,206,893 to Klein et al. (2001) a sealing device, known as the Perclose device, that uses a needle to suture the wound site. This device, however, causes too much trauma to the vessels and pain to patients when the needles are deployed in the artery. Patients feel sharp pain running down their legs when the needles are deployed into the artery tissue. The pain is commonly felt because the nerve in the leg is side by side or close proximity with the leg artery. Thus, this process causes them to jerk or jump when lying down. The other disadvantage is that the Perclose starts off with a 6 French catheter tip to insert into the vessel. However, it gradually turns into an 8 French hub that houses the mechanism for the needle deployment. It is relatively larger in size, causing the wound opening to be bigger than what was originally made, more chances of bleeding, more discomfort to the patient, and more difficulty in closing the wound. Moreover, if the leg artery has some plaque or calcium around the areas where the needles penetrate the artery, the needles will not penetrate that area, thus not securing the suture in place for suturing. If penetration is made, drawing the suture taut would be difficult. The plaque and calcium, being rigid, in the artery or blood vessel would prevent the tissue of the artery or blood vessel from being drawn together. This inability will cause the puncture site to leak or continue to bleed and manual compression be applied. If penetration is not made, another device has to be used because the previous device is unusable at that point. In order to exchange for a second device, an over-the-guidewire technique has to be used. The over-the guide wire technique often requires two people, one person to hold the site of entry to stop the bleeding and the other person to load and insert the new device over a 0.035 inch×135 cm or 145 cm length guidewire. When the exchange is made for the new Perclose, a change in the angle of entry is made because the previous Perclose that couldn't deploy its needles in that site, on many occasions, the previous Perclose has disrupted the integrity of the artery.

U.S. Pat. No. 6,371,974 to Brenneman et al. (2002) is a sealing device that includes a distal end attached to an expansible compression element, which may be an inflatable balloon, a collapsible prong assembly, or a resilient foam pad. When the compression element is placed at the desired position, it gets expanded. Then, pressure is applied to promote hemostasis. This device, however, is seldom used in the field.

U.S. Pat. No. 6,482,179 to Chu et al. (2002) is a sealing device and method with a depth sensing mechanism capable of providing. Furthermore, the method of sealing is more efficient, meaning that no exchange of sheath is needed. This device, however, is not yet being used in the field on a regular basis thus the effectiveness of the device is not known.

Therefore, the common problems among most percutaneous puncture sealing devices are failure to completely seal off the wound site, enlargement of wound site, and/or development of an overly cumbersome process.

### SUMMARY OF INVENTION

A system and method with the option to seal percutaneous punctures of blood vessels. It reduces the multi-step process from intravascular procedures (i.e., angioplasty, atherectomy, stenting, angiography, venous filtration, liquid infusion, dialysis, intravascular ultrasound process, and the like) to sealing in a two-step process. Furthermore, sealing of percutaneous puncture is optional, meaning that the pre-procedural sheath can remain in patients as long as necessary to facilitate the care of the patient. The sheath has reminder or memory markers of entry depth into blood vessels. Additionally, the entry point of sheath is slanted in order to facilitate the entry of sheath into blood vessels. The sealing device comprises of a bio-absorbable inner-wall anchoring member, an outer-wall covering member and a securing member. The use of the anchoring member combined with securing member and covering member will completely cover wound site. The securing member will ensure the permanent adhesion of covering member to outer wall of blood vessel to ensure the non-leaking and rapid clotting of blood, thus promoting faster hemostasis. Furthermore, the sealing members and suture they are tethered and coated with a form of antibiotics or anti-microbial agents to help prevent infection at the wound site and the immediate surrounding area.

Objects and Advantages

The improved sealing device is inserted in the very first step of the intravascular process, thus no exchange of sheath is needed for any intravascular procedure. Furthermore, the new device will have an “option to seal” advantage. If it is so desired not to seal the puncture wound for any reason: e.g., artery disease or blockages in the leg, acute myocardial infarction—current heart attack state, or potent medications that can lower blood pressure rapidly, the device provides a side-port for monitoring blood pressure at the patient's bed side and can be sutured to the patient temporarily. By implementing the pre-procedural sheath before and the “option-to-seal” after an intravascular procedure, the time, number of personnel, mistakes and mishaps, and bleeding from the wound site, and other complications associated with the procedure will be significantly reduced.

The sealing advantage is the assured dislodgement of the anchoring member, which unfolds as it is being pushed out of the beveled-tip pre-procedural sheath from the sealing cartridge device due to the length, width, and depth. Then the gel foam disk (covering member) that will be placed on the outer wall of the blood vessel with a bio-absorbable matrix mesh (i.e., hydrolysate, collagen, or the like) pressing on the gel foam disk against the vessel to completely cover the wound site on the outside of the artery. The covering member is secured by the bio-absorbable matrix mesh, thus preventing the movement of the covering member. The gel foam and bio-absorbable matrix act on different mechanism for hemostasis. The gel foam acts on the blood clotting cascade and the collagen, being a natural ingredient in the body, stimulates the clotting process. Thus, coupling the benefits of both make this closure device superior to other devices that use just one kind. Furthermore, the gel foam disk will promote blood clotting at the site quicker, thus promoting homeostasis at a much faster pace. Additionally, the sealing members and tethering suture are coated with a form of antibiotics or anti-microbial agents, thus preventing possible infection at puncture site and the immediate surrounding area. This is the mechanical advantage and a difference over the other sealing procedures.

### BRIEF DESCRIPTION OF THE DRAWINGS

FIG. 1 is an overall view of the preferred embodiment of the invention.

FIG. 2 is an overall view of the pre-procedural sheath, on embodiment of the invention.

FIG. 3 is an overall Implementation view of the preferred embodiment of the invention.

FIG. 4 is an overall Mid-deployment view of sealing members.

FIG. 5 is a blown-up view of the Mid-deployment status.

FIG. 6 is an overall Final-deployment view of sealing members.

FIG. 7 is a blown-up view of the Final-deployment status.

FIG. 8 is a view of all sealing members inside the sealing device.

FIG. 9 is a view of sealing members outside the delivering tube of sealing device.

FIG. 10 is a view of sealing members completely deployed from sealing device.

### DETAILED DESCRIPTION OF THE PREFERRED EMBODIMENT

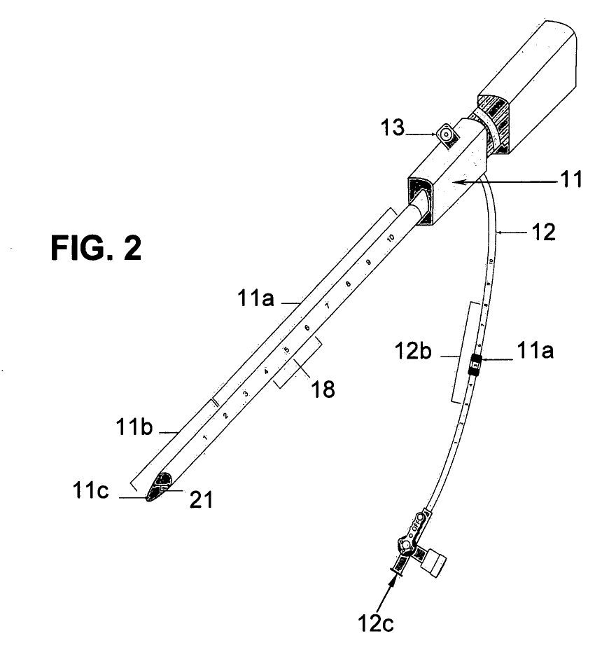

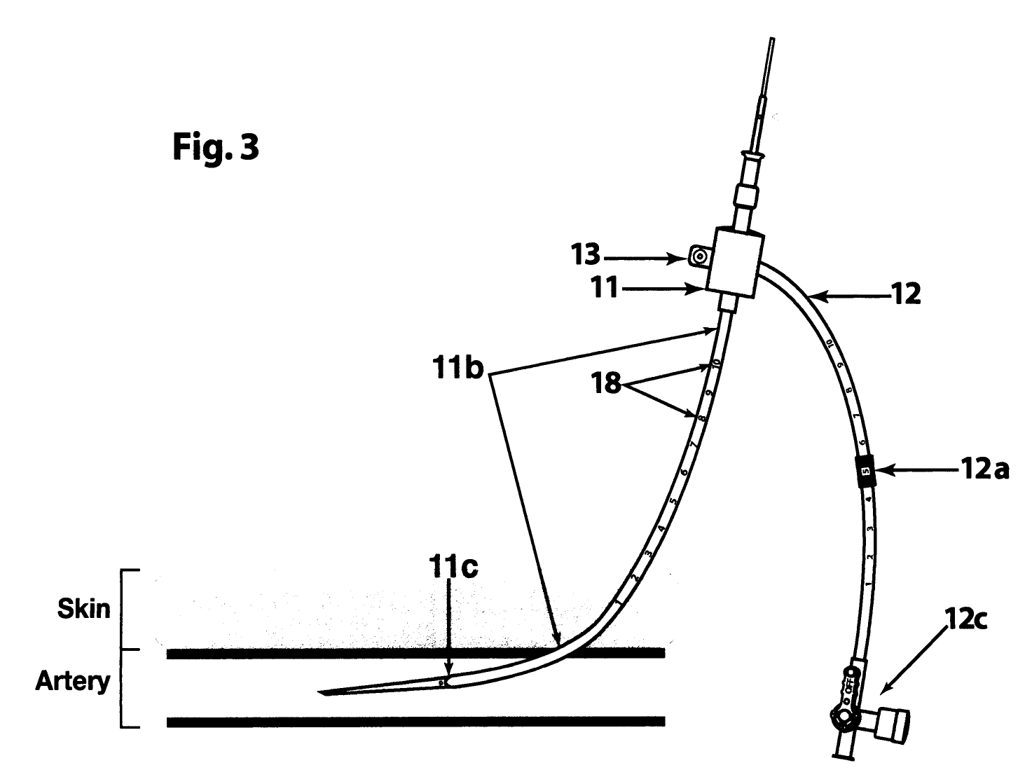

The two main parts of the invention are: one, the pre-procedural sheath, illustrated in FIG. 2; and two, the sealing device, illustrated in FIG. 8, 9, & 10.

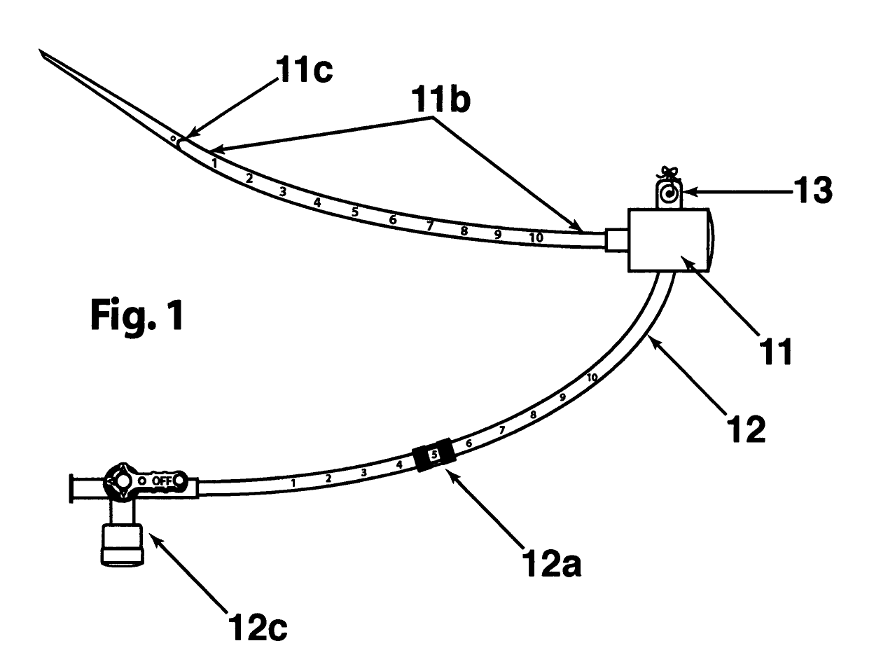

The typical and conventional intravascular surgical procedure (i.e., angioplasty, atherectomy, stenting, angiography, venous filtration, liquid infusion, dialysis, intravascular ultrasound process, and the like) starts with the insertion of a percutaneous 18 gauge needle (not shown) through the skin into the artery. Then, a 0.035 inch guide wire (not shown) is inserted through the needle into the artery to the desired location in the artery. Once the 0.035 inch guide wire is in place, the needle is removed. The improved sheath (FIG. 1) is passed over the 0.035 inch guide wire into the artery. The entry point 11c of the distal portion 11b of sheath is slanted at an angle between fifteen to sixty degrees in order to facilitate a smooth entry of sheath into a blood vessel. Having a beveled edge on the improved sheath makes an easy transition through the skin and into the arterial muscle wall. This bevel is more beneficial when the artery has been accessed numerous times and scar tissue has developed on the artery where the entry point is made. The bevel ensures a smoother pass into the artery compared to standard level introducer sheaths in industry. In addition, the shape will facilitate the expulsion of other sealing members such as a rigid anchor, a sealing disk, or the like when deployed.

The sheath's diameter 21 can range somewhere between 5.25 French to 9.25 French with 1 French increments (i.e., 5.25 F, 6.25 F, 7.25 F, 8.25 F, and 9.25 F), depending on the need of the intravascular procedure (FIG. 2). The benefits and advantage for having quarter sizes for sheath's diameter 21 are two-fold. The first benefit is when comparisons of blood pressure of the heart are needed to be compared to the systemic blood pressure in the aorta. This type of valve pressure measurement is crucial to determine the difference between valve surgery and medical treatment. The quarter-size difference will allow simultaneous monitoring of blood pressure to be performed during an intravascular procedure. The conventional method used today is the use of whole sized sheath introducers; e.g., a 6 French introducer sheath and a 6 French catheter. If simultaneous pressures are desired, a 1 French size difference is needed between the standard introducer sheath and a catheter that is inserted through the hemostatic valve assembly 1 to the heart chambers; e.g., a 7 French introducer sheath and a 6 F catheter. The 1 French size difference will allow simultaneous pressures to be measured. A bigger sheath is least likely chosen because the conventional thought is the bigger the sheath, the bigger the hole; and the bigger the hole, the more chance of bleeding complications. It is often the case that the smaller the equipment you can get away with to perform the intravascular procedure, the better. The second benefit is to allow for blood drawing during the procedure to ensure blood clotting time is extended by use of thrombolitic therapy medications to dissolve and prevent any blood clot material that might go to the brain, heart, or lung.

The sheath has calibration markings 18 (i.e., numbers, letters, color coded symbols, or the like) on distal member 11c to identify the improved sheath's exact placement in the blood vessel (FIG. 2). Then, the exact mark is transferred to the side-port 12 by sliding the movable marker member 12a (i.e., rubber or metal ring or clip, or the like) to the appropriate matching side-port calibration markings 12b (i.e., numbers, letters, color coded symbols, or the like) on side-port 12. The movable reminder member 12a on side-port calibration markings 12b serves as a conspicuous and secured reminder of the placement depth of sheath in blood vessel. Then the guide wire is removed leaving the improved sheath in place. A catheter or other intravascular instrument (not shown) is then inserted through the sheath. Once the intravascular procedure has been completed, the intravascular instrument or catheter is removed.

However, if the improved sheath remains in the patient, the side-port 12 which is fused to the hemostatic-valve assembly 11 can be hooked up at 12c to monitor femoral artery pressure (FIGS. 2 & 3). This procedure measures pressure via a transducer and performed to monitor femoral (systemic) artery pressure instead of using a blood pressure cuff on the arm (not shown). On the other side of the hemostatic-valve assembly 11 is a suture tab 13. The improved sheath could be sutured to the patient's skin through the suture tab, thus holding the improved sheath in place (FIG. 1), thus sealing of wound site is only an option and is not mandatory.

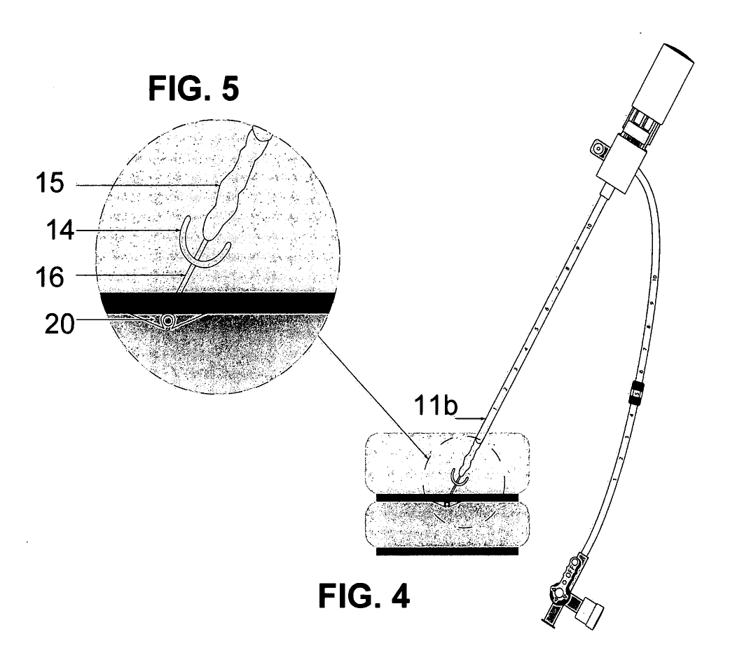

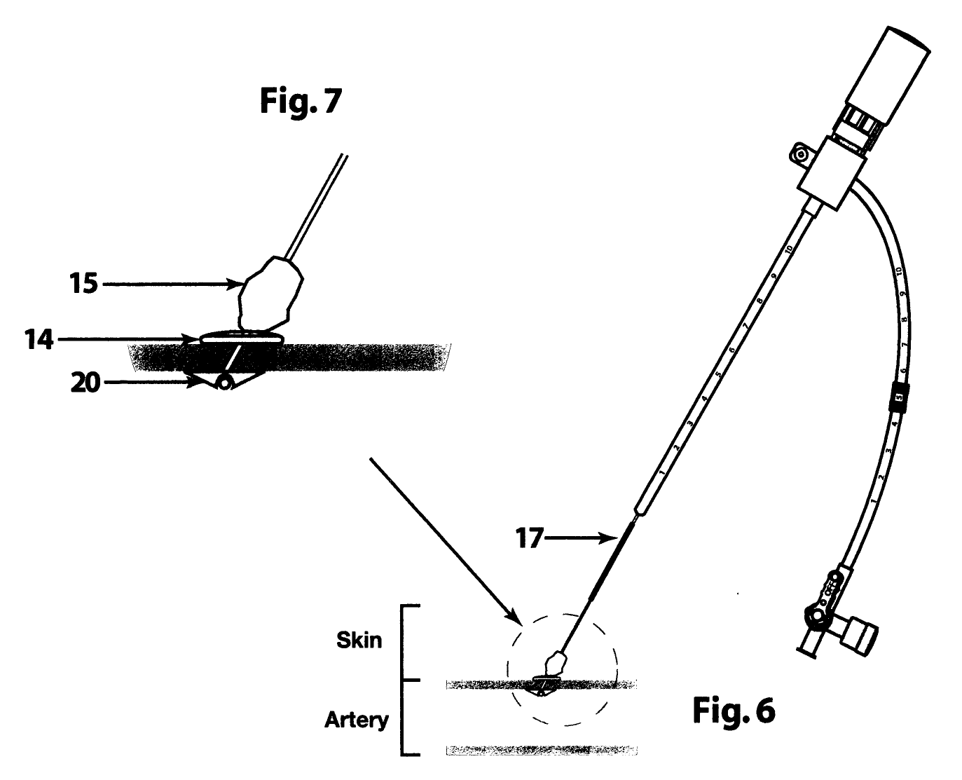

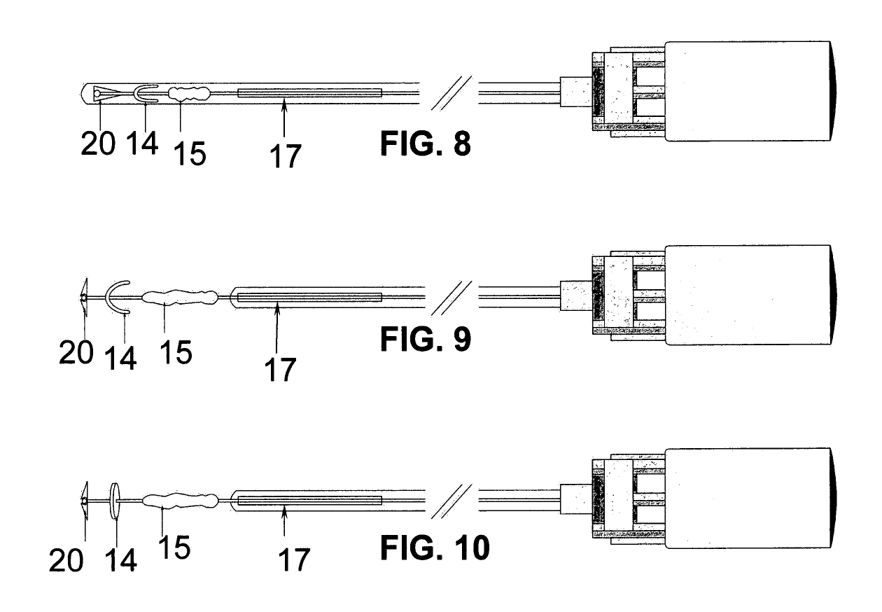

Once sealing of the puncture site is decided, the sealing cartridge device (FIGS. 4, 5, 6, 7, 8, 9, & 10) is introduced into the puncture site in the artery wall by way of the hemostatic-valve assembly 11 after the pre-procedural sheath is brought to the same calibration that was transferred to the side-port calibration markers from the initial measurement. The sealing cartridge device can vary in diameter (i.e., 5 French to 9 French with 1 French increments) to fit the diameter of the pre-procedural sheath. The sealing device comprises of a bio-absorbable inner-wall anchoring member, an outer-wall covering member 14 (i.e., gel foam disk or the like) and securing member 15 (i.e., bio-absorbable matrix mesh such as collagen, hydrolysate, or the like) of outer-wall covering member. The anchoring member 20 is folded in half when positioned within the sealing device with the suture 16 attached at its center of the anchor (FIGS. 8, 9, & 10). The anchoring member 20, however, unfolds as it being pushed outside the sealing cartridge device, serving as an anchor inside the vessel. The beveled shape of the pre-procedural sheath will ensure the unfolding of the anchoring member. This unfolding will cause the anchor to deploy inside the lumen of the blood vessel. The shapes of the anchoring member vary in length, width, and depth, so long as it can be pushed out of the sealing device with ease and not interfere with the flow of blood in the vessel. For example, the anchoring member can be of a rod-like shape to a narrow oval shape. In addition, the variable sizes of the anchoring member can be used for the different pre-procedural sheath sizes; and complements the pre-procedural sheaths used for the blood vessel that was accessed.

The covering member 14 should be of sufficient size to completely cover wound site (FIGS. 4, 5, 6, & 7). The securing member 15 is approximately of a quantity standard in the industry that can ensure the security of the placement of the covering member 14. Furthermore, the quantity of securing member 5 has to be absorbed by the body within 4-6 weeks, a typical period of time accepted in the field. The gel foam and bio-absorbable matrix act on a different mechanism for hemostasis. The gel foam acts on the clotting cascade in the blood and the collagen is an ingredient that stimulates the clotting process. So, coupling the benefits of both makes a closure device superior to others devices that just use one kind.

However, the outer-wall covering member 14 and the securing member 15 can be used with existing sealing members of other closure devices that have an inner wall and/or between wall sealing members. The outer-wall covering member 14 is placed on the outer wall of the artery or blood vessel. Then, the securing member 15 is introduced on top of the outer-wall covering member 14 to hold the covering member in place (FIGS. 4, 5, 6, & 7). Both covering member 14 and securing member 5 are tethered on an antibiotic or povidone-iodine, as in Betadine® Microbicides, coated suture 16. However, antibiotics can also be delivered to the wound site with other sealing members such as covering member 14, securing member 15, or other sealing members found presently in other closure devices. The securing member 15 is packed on to the covering member 14 using a compaction tube 17.

1. A means to beginning an intravascular procedure, comprising:
a) a side-port with matching calibration markings as distal member of sheath and a movable reminder member calibration markings on distal member of sheath,
b) a diameter with sufficient difference from a standard catheter to allow monitoring blood pressure while simultaneously performing the intravascular procedure, and
c) a slanted entry on distal member,
whereby said sheath will allow accurate recording of sheath's position in blood vessel, smoother sheath entry into blood vessel, and simultaneous drawing of blood pressure from the heart for certain medical diagnostic procedures.

2. A means to seal puncture wounds in a blood vessel, comprising:
a) an inner-wall anchoring member that unfolds from its center as it is being deployed,
b) an outer-wall covering member with sufficient surface area to completely cover wound site,
c) a securing member in some form of a bio-absorbable mesh,
d) a diameter with sufficient difference from said sheath's diameter to allow easy movement of said sealing means, and
e) a means to deliver antibiotics or anti-microbial agents to wound site and its immediate surrounding,
whereby said sealing means ensure easy and guaranteed deployment of said anchoring member and complete coverage of wound opening due to the three layers of sealing and lower the chances of infections at wound site to promote faster healing and hemostasis.

3. A method to begin an intravascular procedure, comprising:
a) transferring of marking calibrations on said sheath is to the said matching marking calibrations on side-port,
b) sealing of wound site is optional and not mandatory, thus the sheath can be left in patients as long as needed,
c) monitoring blood pressure from the heart simultaneously performing the intravascular procedure because of sheath's varying diameters, and
d) inserting sheath with ease through tissues and blood vessels because of the slanted entry point on distal end of sheath.

4. A method for sealing wound site of blood vessels, comprising:
a) placing the anchoring member inside the blood vessel
b) placing of securing member directly on top of outer-wall covering member,
c) sealing wound site without changing the pre-procedural sheath, and
d) a method to delivering antibiotics or anti-microbial agents to the wound site and immediate surrounding area.

5. The pre-procedural sheath in claim 1 wherein said sheath facilitates and/or is used to implement a vascular closure device to seal and close a wound entry site in a blood vessel.
